(19) United States
(12) Patent Application Publication
(10) Pub. No.: US 2005/0171077 A1
(43) Pub. Date: Aug. 4, 2005

(54) Compositions containing piperacillin and tazobactam

(75) Inventors: Mark Edward Ruppen, Garnerville, NY (US); Yu Fen Wang, Demarest, NJ (US); Sam Mattackal George, Wayne, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/045,435

(22) Filed: Jan. 28, 2005

Related U.S. Application Data
(60) Provisional application No. 60/540,910, filed on Jan. 30, 2004.

(51) Int. Cl. A61K 31/43; A61K 31/407

(52) U.S. Cl. 514/192; 514/210.09

(57) ABSTRACT

The invention pertains to pharmaceutical compositions of Zosyn® having substantially free or reduced levels of galactomannan and processes to prepare said pharmaceutical compositions.

This application claims priority from copending provisional application Ser. No. 60/540,910 filed on Jan. 30, 2004 the entire disclosure of which is hereby incorporated by reference.

### FIELD OF THE INVENTION

The invention relates to pharmaceutical compositions of Zosyn® substantially free of galactomannan.

### BACKGROUND OF THE INVENTION

Zosyn® is an antibiotic marketed product in the United States and Tazocin brand in many foreign countries which contains piperacillin sodium and tazobactam sodium. The product is disclosed in U.S. Pat. No. 4,562,073. U.S. Pat. Nos. 4,477,452 and 4,534,977 disclose a lyophilized form of piperacillin.

Zosyn® is an antibiotic which is used in the treatment of moderate to severe infections. In particular, Zosyn® is used in the treatment of moderate to severe infections caused by piperacillin-resistant, piperacillin/tazobactam-susceptible beta-lactamase-producing strains of microorganisms in conditions such as nosocomial pneumonia due to Staphylococcus aureus; intra-abdominal infections, specifically appendicitis (complicated by rupture or abscess) and peritonitis due to Escherichia coli, skin and skin structure infections, including cellulites, cutaneous abscesses and ischemic/diabetic foot infections due to Staphylococcus aureus; and gynecologic infections, specifically postpartum endometritis or pelvic inflammatory disease due to Escherichia coli. The seriousness of these infections highlights the need for a readily available and dependable treatment.

Medicaments are formulated into not only emulsions, suspensions or solutions, but also as lyophilized preparations to be reconstituted before use. Advantageously, lyophilized preparations are stable, can be stored and are easily reconstituted. Moreover lyophilized preparations may be kept sterile and essentially free of insoluble matter.

Zosyn® is available as a powder (lyophilized product) which is reconstituted by addition of a compatible reconstitution diluent prior to intravenous administration. Zosyn® has been found to contain trace amounts of galactomannan which is a carbohydrate polymer derived from fungal cell walls and formed in fermentation processes. The presence of galactomannan is shown to interfere and provide false positives in certain diagnostic tests for invasive aspergillosis (IA). Although present, galactomannan does not create an increased health risk to the patient.

The disadvantages of the presence of galactomannan in pharmaceutical compositions of Zosyn® are overcome by the present invention.

### BRIEF SUMMARY OF THE INVENTION

Zosyn® has been found to contain trace amounts of galactomannan, a carbohydrate polymer derived from fungal cell walls. However, though present, galactomannan does not create an increased health risk to the patient.

Invasive aspergillosis (IA) is a fatal fungal infection most frequently seen in immuno compromised patients. The presence of circulating aspergillus galactomannan antigen in serum is indicative of invasive aspergillosis (IA), a fatal fungal infection. Immuno compromised patients frequently are subjected to prophylactic treatment with Zosyn® to prevent bacterial infections. The diagnosis of invasive aspergillosis in patients is often done based on serological methods by detecting the presence of aspergillus galactomannan. The presence of trace amounts of galactomannan in Zosyn®, however leads to false positive test results for IA when using certain diagnostic kits. The removal of galactomannan from Zosyn® has the advantage of eliminating or decreasing the potential for false positive diagnostic test results for IA when using said kits.

The present invention provides to the art a new pharmaceutical composition of premixed piperacillin or piperacillin-tazobactam which avoids the presence of galactomannan and is useful for the treatment or control of bacterial infections by parenteral administration, the composition comprising effective amounts of (a) piperacillin or a pharmaceutically acceptable salt thereof (normally as piperacillin sodium), and (b) tazobactam or a pharmaceutically acceptable salt thereof (normally as tazobactam sodium). The pharmaceutical composition according to the invention may be (A) in the form of a powder that can be reconstituted by addition of a compatible reconstitution diluent prior to parenteral administration, (B) in a form ready to use for parenteral administration or (C) in a frozen form which can be thawed and is ready to use for parenteral administration. The composition of the invention is provided substantially free of galactomannan.

The invention further includes:
A process for preparing a lyophilized pharmaceutical composition which is substantially free of galactomannan which comprises the steps of:
   a) dissolving piperacillin, and tazobactam, in an aqueous solvent forming a solution and adjusting the pH to about 6.5;
   b) filtering the solution through a cut off filter;
   c) collecting a filtrate;
   d) cooling the filtrate to a temperature below −35° C. in a lyophilizer;
   e) evacuating the lyophilizer to a pressure of about 300 μM Hg (micrometers of mercury) (40 pascals) and heating the lyophilizer to about +5° C.;
   f) maintaining the temperature and pressure for a sufficient time to remove water from the aqueous solvent forming a lyophilized solid;
   g) drying the lyophilized solid at about +45° C.

The invention also includes a process for the manufacture of a pharmaceutical composition in the form of a powder that can be reconstituted by addition of a compatible reconstitution diluent prior to administration to a mammal or in the form of a frozen composition which when thawed can be diluted with a compatible diluent prior to administration to a mammal which process comprises freezing or freeze-drying a solution substantially free of galactomannan containing effective amounts of (a) piperacillin or a pharmaceutically acceptable salt thereof, (b) tazobactam or a pharmaceutically acceptable salt thereof in an aqueous vehicle.

### DESCRIPTION OF PREFERRED EMBODIMENTS

The present inventive composition offers an advantage over other forms of piperacillin and piperacillin-tazobactam for administration. In particular, the invention provides a composition which is substantially free of galactomannan. Without the presence of galactomannan in the composition of Zosyn® there is a lack of interference and false positive test results with antibody tests which are used for the determination of invasive aspergillosis. Critical to the removal or reduction of the galactomannan is the use of an appropriate cut off filter of about 3 kD mw to about 10 kD mw. The galactomannan collects on the filter and the piperacillin or piperacillin-tazobactam pass through the filter and are in the collected filtrate. Preferred is a molecular weight cut off filter of about 3 kD. More preferred is a cut off filter of about 5 kD.

The removal or reduction of galactomannan proceeds in the following manner: an aqueous solution of Zosyn® at about (10 mg/ml) is prepared. The solution is applied to a series of micro centrifuge filter devices(Pall Life Sciences) and the filters are centrifuged at 10,000×g. This procedure forces the solution through the ultrafiltration membrane. Solutes are separated by the membrane based on molecular weight. Low molecular weight materials, such as piperacillin and tazobactam, pass through the ultrafiltration membrane (filtrate) while materials with a molecular weight greater than the membrane cut off are effectively retained by the filter (retentate). Galactomannan is reported to have a high molecular weight of 25,000 to 75,000; piperacillin and tazobactam have low molecular weights<1000. When a solution of Zosyn® containing galactomannan is applied to a 3000 mw cut off filter and spun, the galactomannan is found in the retentate (R). The filtrate (F) contains the piperacillin and tazobactam components and the filtrate tests negative for galactomannan. Similar results are found with a 5-kD membrane. Results found using a 10-kD cut off filter show that minor amounts of galactomannan are found in the filtrate. Importantly though there is no loss in strength of the piperacillin and tazobactam in the filtrate when compared to the starting material. In typical experiments, where the progress is followed by high pressure liquid chromatography (HPLC) the following results are obtained after ultrafiltration.
1. Zosyn®
   Recovery of Tazobactam—100.3%
   Recovery of Piperacillin Monhydrate—99.0%
2. Piperacillin—100.1%
3. Ampicillin—99.8%

This process is easily adapted to a production scale for commercial operations using currently available ultrafiltration (UF) devices and membranes.

Galactomannan can be effectively removed from Zosyn® solutions by ultrafiltration. Work has shown that filtration through the appropriate molecular cut off membrane filter separates the high molecular weight galactomannan from the low molecular weight Zosyn® components. Further removal of galactomannan and increased recovery of piperacillin and tazobactam may be further accomplished in commercial operations using diafiltration with membrane filters as a portion of the cut off filter ultrafiltration. The membrane filters in diafiltration retain the galactomannan and allow the Zosyn® components to pass through and be collected in the filtrate. Galactomannan may also be removed from 6-aminopenicillanic acid (6-APA) and ampicillin by the appropriate membrane filter.

Experimental Protocol

TITLE: Evaluation of Zosyn®, active pharmaceutical ingredient (API's) and other antibiotics for the presence of galactomannan using BIO-RAD Platelia® Aspergillus EIA method

1. Purpose

The purpose of this protocol is to describe the experimental design for evaluation of different lots of Zosyn®, APIs and other antibiotics for the presence of galactomannan antigen using BIO-RAD Platelia® Aspergillus EIA kit.

2. Materials and Equipment

2.1 Samples and Reagents

1. Samples

Zosyn® 2.250 g/vial

Zosyn® 4.5 g/vial

Tazoxil 4.5 g (generic Zosyn®) from Brazil

Tazac 4.5 g (generic Zosyn®) from India

Piperacilina Tazobactam 4.5 g (generic Zosyn®) Richet (Argentina)

Other products are included in this protocol to evaluate its response in BIO-RAD Platelia® Aspergillus EIA diagnostic kit.

2. Platelia® Aspergillus EIA (BIO-RAD, Redmond, Wash.), No. 62793 (96 Test Kit) or No. 62794 (480 Test Kit)

2.2 Equipment

1. Micro Plate reader: Dynex MRX ELISA plate reader

2. Ultrawash II Automatic washer/Aspirator, Dynex

3. Biosafety cabinet

4. Boiling water bath

5. Incubator

6. Vortex agitator

7. Sterile tubes, sterile gloves and sterile pipette tips

8. Micropipette

3. Environmental Control

Preparation of reagents, sample and sample dilutions will be done under aseptic conditions in a Biosafety cabinet.

4. Test Site

Zosyn®

Experiments conducted at the Chemical Process Development Biochemistry Laboratory, Wyeth Research, Pearl River, N.Y.

5. Assay Principal and Procedure

5.1 Assay Principal

The Platelia® Aspergillus EIA is a one-stage immunoenzymatic sandwich microplate assay used for the detection of galactomannan in human serum. A rat monoclonal antibody EBA-2 is used to capture the antigen, which is then, detected using a perioxidase conjugated-antibody. The absorbance value of the sample is compared to the absorbance value of the “cut off” control thus determining the index/relative concentration of galactomannan.

5.2 Procedure

Refer to the BIO-RAD Platelia® Aspergillus EIA kit user manual for reagent preparation, step by step assay procedure and safety instructions on handling the reagents and samples.

Sample preparation: Reconstitute in water for injection (WFI)USP grade (United States Pharmacopeia) or any other appropriate diluents and make dilutions at desired concentrations.

The dilution of the sample may be changed based on the results of the proceeding experiments.

6. Experimental Design

6.1 Product Evaluation

1. Evaluation of Zosyn®

Analyze vials of Zosyn® at desired concentrations in water for injection (WFI)/phosphate buffer solution (PBS) or other appropriate matrix.

2. Evaluation of active pharmaceutical ingredients(APIs)

Analyze vials of piperacillin and tazobactam, and any other available intermediates in water for injection (WFI)/phosphate buffer solution (PBS) .

3. Generic Zosyn® and/or other antibiotics

Analyze other available generic Zosyn®/antibiotics at desired concentrations in WFI/PBS.

6.2 Filtration Studies

Zosyn®

1. Filter reconstituted samples using appropriate molecular weight cut off spin filters and test the filtrate at desired concentrations. Evaluate other studies on filtration capabilities as appropriate.

3. Acceptance Criteria

Cut-off Control: The optical density (OD) 450 of each (2) Cut-off Control Serum well must be between 0.3 and 0.8. Each individual value should comply the specification. The Mean Cut off Control is the average of the two well readings. (see BIO-RAD kit instructions). Positive Control: The index of the Positive Control Serum must be greater than 2. Negative Control: The index of the Negative Control Serum must be less than 0.4. Failure of any of the controls to meet the criteria renders the assay invalid. To determine the index for experimental samples, divide the absorbance (OD450) of the test sample by the Mean Cut-off Control. An index greater than 0.5 is considered a positive result. An index less than 0.5 is considered a negative result.

### 4. REFERENCES

Platelia® Aspergillus EIA manual (BIO-RAD, Redmond, Wash.).
I=OD Positive Control (R5)>2
Mean Cut-off Control OD
I=OD Negative Control (R3)<0.4
Mean Cut-off Control OD
   Zosyn® (Pipercillin/Tazobactam) Strength and Identification in Aqueous Samples by High-Performance Liquid Chromatography
   1. Outline of Method

A portion of the sample of Zosyn® is dissolved and diluted with dilution solvent then chromatographed on a reversed phase column (USP 23 NF18, Supp. 6, p. 3722). The Piperacillin, and Tazobactam strengths are determined by comparing the respective peak responses in the sample preparation chromatogram to those of the standard chromatograms obtained concomitantly. Piperacillin, and Tazobactam are identified by comparing the retention times of the respective peaks in the sample preparation chromatogram with those of the respective peaks in the standard preparation chromatograms. The method reporting limit for Piperacillin is 0.16 μg/mL for the solution injected. The method reporting limit for Tazobactam is 0.077 μg/mL for the solution injected.

2. Special Equipment

Chromatographic Column—Length about 25 cm, inside diameter about 4.6 mm, packed with Phenomenex Luna C18 (2), 5 μm size particles.

NOTE: Columns of lengths 150 mm to 300 mm may be used provided the system suitability requirements are met.

Pump—Constant flow pump capable of operating at pressures up to 5000 psi.

Detector—Ultraviolet spectrophotometric detector capable of operating at 220 nm with a sensitivity of about 1.0 absorbance units full scale.

Injector—Any manual injector or auto-injector capable of reproducible injections and maintaining a sample tray temperature of 5° C.

Integrator—Electronic integration is preferred.

Recorder—Optional. A recording device matched to the operating output voltage of the detector.

Membrane Filter—Pore size 0.45 μm, Nylon-66 membrane filters.

Column Temperature Controller—Capable of maintaining a column temperature of 30° C.

3. Reagents and Materials

Methanol—HPLC grade.

Sodium Phosphate, Monobasic—(NaH₂PO₄) Reagent grade.

Tetrabutylammonium Hydroxide 0.4 M—Reagent grade.

Phosphoric Acid—85%, Reagent grade.

Water—Suitable for HPLC.

0.2 M Monobasic Sodium Phosphate Buffer Solution—Weigh 27.6 g of monobasic sodium phosphate and dilute to 1 L with water.

20% Phosphoric Acid Solution—Dilute 23.5 mL of 85% phosphoric acid to 100 mL with water and mix.

2% Phosphoric Acid Solution—Dilute 2.4 mL of 85% phosphoric acid to 100 mL with water and mix.

Dilution Solvent—Mobile phase.

Mobile Phase—Measure 447 mL of water, add 100 mL of 0.2 M monobasic sodium phosphate buffer solution, pipet 3.0 mL of tetrabutylammonium hydroxide and add 450 mL of methanol. Mix. Cool to room temperature. Adjust the pH of the solution to approximately 5.6 with the 20% phosphoric acid solution and then to 5.50±0.02 with the 2% phosphoric acid solution. Filter through a 0.45 μm pore size membrane filter, if necessary. Degas if necessary.

Piperacillin Reference Standard—Of known strength (S).

Tazobactam Reference Standard—Of known strength (S).

4. Equipment Preparation

1. Set the detector wavelength to 220 nm and the sensitivity at about 1.0 absorbance units full scale. (The sensitivity setting may vary depending on the apparatus used).

2. Set the flow rate at 0.8 mL per minute (0.5 to 1.2 mL per minute is acceptable).

3. Set column temperature controller to 30° C.

4. Set injector/autosampler temperature controller to 5° C.

5. Pump mobile phase through the column until a stable baseline is obtained (usually about 15× column volume).

5. Standard Preparation

1. Accurately weigh about 24 mg of Tazobactam reference standard, and 20 mg of Piperacillin reference standard into 2 separate 50 ml volumetric flasks.

2. Dissolve the standards with a few drops of methanol (sonicate if necessary) and dilute the Tazobactam to volume with dilution solvent. This is the Tazobactam standard stock solution.

3. Pipet 5.0 mL of the Tazobactam standard stock solution into the Piperacillin flask. Dilute to volume with dilution solvent and mix. This is the Piperacillin/Tazobactam standard preparation. (approximately 400 and 48 μg/mL, respectively). These are for single point standard calculations.

4. (This step is required only when vehicle/control samples are being assayed). Pipet 2.0 mL of the Piperacillin/Tazobactam (400/48 μg/mL) standard preparation into a 100 mL volumetric flask and dilute to volume with dilution solvent. Pipet 2.0 mL of this solution each into 100 and 25 mL volumetric flasks and dilute to volume with dilution solvent. These are the reporting limit standard preparations for Piperacillin and Tazobactam, respectively, (approximately 0.16 μg/mL of Piperacillin for the first solution and 0.077 μg/mL of Tazobactam for the second solution). For each of these preparations only the relevant concentration is used.

NOTE 1: Linearity for Piperacillin has been established from 100 to 500 μg/mL. Linearity for Tazobactam has been established from 10 to 100 μg/mL. Proportionately smaller or larger standard weights may be taken, provided that any subsequent dilutions are adjusted accordingly to yield standard preparation concentrations within the linear range. If this is done, suitable adjustments must be made to the calculations.

NOTE 2: Other dilution schemes are possible provided that the final dilutions and injected concentrations are within the linear range. If this is done, suitable adjustments must be made to the calculations.

6. Sample Preparation

Based on the claimed concentrations of the sample, make necessary dilutions in dilution solvent to obtain a sample solution concentration near the single point standard concentrations for Piperacillin and Tazobactam (approx. 400 and 48 μg/mL, respectively). For the typical ±2 mL pre-measured sample, quantitatively transfer the entire sample. Rinse vial, vial cap, and outside of vial neck adding the rinses to the dilution flask.

If necessary, vortex the sample vial during rinsing to remove all of the sample. Dilute to volume with dilution solvent and mix well. Any subsequent dilutions should also be made in dilution solvent. Samples should be processed one at a time to minimize the time before being injected.

NOTE 1: Non-typical samples may require an alternate preparation procedure. For example, the sample volume or concentration may necessitate that an aliquot be taken.

NOTE 2: Dilute vehicle/control samples 2:10 for the typical 2 mL sample for Piperacillin. For Tazobactam further dilute the sample 2:10.

If samples are pre-weighed, the initial sample volume should be calculated using the density as follows:
(mL) volume Sample=(g) mass Sample/(g/mL) Density
   7. System Suitability

1. After a stable baseline has been obtained, inject 10 μL of the Piperacillin/Tazobactam standard preparation three times and obtain a chromatogram of piperacillin/tazobactam reference standard. These injections are used for System Suitability and Calculations.

2. Calculate the capacity factor, k′, of Piperacillin. The capacity factor must be 3.5 or higher. If not, prepare fresh mobile phase or replace the column.

NOTE: The ta value (the retention time of an unretained peak) may be estimated by dividing 60 percent of the column volume by the flow rate in mL/minute. For the Phenomenex column specified, the ta estimation is 2.5 mu(flow rate in mL/minute).

3. Calculate the column tailing factor, T, as directed in the USP. The column tailing factor must not be more than 1.5. If more, repair the chromatographic system and/or replace the column.

4. Calculate the theoretical plates, N, as directed in the USP. The value of N must be greater than or equal to 3000. If less, decrease the flow rate within the allowable range, replace the column and/or repair the chromatographic system.

5. Calculate the RSD for the three replicate injections of Piperacillin. The RSD must not be more than 2.0%.

8. Procedure

A. Strength

1. (This step is required only when vehicle/control samples are being assayed). At some point during the assay, inject 10 μL of dilution solvent to obtain a blank chromatogram.

Inject 10 μL of the sample preparation(s) and the reporting limit standard preparations and obtain the response(s) at the retention time of the peak of interest.

2. Inject 10 μL sample preparation and obtain the responses of the peaks of interest.

B. Identification

1. Inject 10 μL each of the Piperacillin/Tazobactam and obtain the retention time of the respective peaks.

2. Inject 10 μL of the sample preparation and obtain the retention time of the respective peaks.

9. Calculations

A. Strength

1. Calculate the Piperacillin/Tazobactam concentration of the standard preparation from the following equations:
mg of Piperacillin/ml=(Wr)(S)/(50)
mg of Tazobactam/ml=(Wr)(S)(V1)/(50)(V2)
Where:

Wr=weight of the respective reference standard, mg

S=strength of the respective reference standard, decimal

V1=volume of the standard stock solution used to make the standard preparation, mL

50=volume of the standard stock solution or standard preparation, mL

V2=volume of the standard preparation, mL

2. For Piperacillin and Tazobactam:

Calculate the strength(s) from the equation:
mg Piperacillin or Tazobactam/mL=(Cs)(Rspl) (Dspl)/(Rstd)
Where:

Cs=concentration of the respective standard from 1 above, mg/mL

Rspl=response for sample preparation

Dspl=dilution factor for the sample preparation

Rstd=average response for the respective standard preparation

B. Identification

1. Calculate the relative retention value (Rr) of the respective peak in the sample preparation chromatogram using the expression:
Rr=Rt of the respective peak, from the sample chromatogram/Rt of the respective peak, from the standard chromatogram

Rt=retention time, minutes

2. Report the identity as positive if:
Rr is 1.0±0.05, otherwise report the identity as negative.
   10. Reporting Limit

The reporting limit for Piperacillin for this method is 0.16 μg/mL for the solution injected.

This is 0.8 μg/mL for a 2 mL vehicle/control sample diluted 2 mL to 10 mL. The reporting limit for Tazobactam for this method is 0.077 μg/mL for the solution injected. This is 1.92 μg/mL for a 2 mL vehicle/control sample diluted 2 mL to 10 mL then 2 mL to 10 mL again.

Filtration Study

Zosyn® (typical commercial sample) is dissolved in water at 100 mg/ml. Piperacillin is dissolved in saturated sodium bicarbonate at 100 mg/ml. Zosyn® and piperacillin are diluted to 10 and 1 mg/ml using USP water. Zosyn® (300 μl) at 10 and 1 mg/ml as well as piperacillin are transferred to the nanosep spin device with 10 kD or 3 kD molecular weight cut-off filters. Samples are placed in a eppendorf centrifuge and centrifuged for 10 minutes at 10,000 rpm. At the end of the centrifugation, samples were collected in the pass-through. The retained galactomannan in the upper part of the nanosep spin device are resuspended with 300 μl of water for assay. Typical results are displayed in the following Examples 1-4. Optical density (OD) for galactomannan are displayed for each example, as well as the determined index of experimental samples.

Results:

Neg. CTL: 0.078, index=0.14

C-O CTL: 0.534, 0.554, mean optical density (OD)=0.544

Pos CTL: 2.009, index 3.69

### EXAMPLE 1

### 10K(10 kD) Filter

| | | | | Index of |
|---|---|---|---|---|
| Experimental | | | | Experimental |
| Samples | OD1 | OD2 | Mean OD | Samples |
| Zosyn ®, no | over | over | over | |
| filtration, | | | | |
| 10 mg/ml | | | | |
| Zosyn ®, no | 1.135 | 1.102 | 1.119 | 2.056 |
| filtration, | | | | |
| 1 mg/ml | | | | |
| Zosyn ®, 10 mg/ml, | 2.173 | 2.152 | 2.163 | 3.975 |
| 10K, | | | | |
| (R)* | | | | |
| Zosyn ®, 10 mg/ml, | 0.264 | 0.27 | 0.267 | 0.491 |
| 10K, (F)** | | | | |
| Zosyn ®, 1 mg/ml, | 0.263 | 0.264 | 0.264 | 0.484 |
| 10K, | | | | |
| (R)* | | | | |
| Zosyn ®, | 0.046 | 0.045 | 0.046 | 0.084 |
| 1 mg/ml, 10K, | | | | |
| (F)** | | | | |

*(R) is the retentate (retained on the filter)
**(F) in the filtrate

### EXAMPLE 2

### 3K(3 kD) Filter

| | | | | Index of |
|---|---|---|---|---|
| Experimental | | | | Experimental |
| Samples | OD1 | OD2 | Mean OD | Samples |
| Zosyn ®, 10 mg/ml, | over | over | over | |
| 3K, (R)* | | | | |
| Zosyn ®, 10 mg/ml, | 0.041 | 0.04 | 0.041 | 0.074 |
| 3K, (F)** | | | | |
| Zosyn ®, 1 mg/ml, | 0.748 | 0.791 | 0.770 | 1.415 |
| 3K, (R)* | | | | |
| Zosyn ®, | 0.042 | 0.045 | 0.044 | 0.080 |
| 1 mg/ml, 3K, | | | | |
| (F)** | | | | |

*(R) is the retentate (retained on the filter)
**(F) in the filtrate

### EXAMPLE 3

### 10K(10 kD ) Filter

| | | | | Index of |
|---|---|---|---|---|
| Experimental | | | | Experimental |
| Samples | OD1 | OD2 | Mean OD | Samples |
| Piperacillin, | 1.892 | 1.953 | 1.923 | 3.534 |
| no filtration, | | | | |
| 10 mg/ml | | | | |
| Piperacillin, | 0.477 | 0.463 | 0.470 | 0.864 |
| no filtration, | | | | |
| 1 mg/ml | | | | |
| Piperacillin, | 2.031 | 2.131 | 2.081 | 3.825 |
| 10 mg/ml, | | | | |
| 10K, (R)* | | | | |
| Piperacillin, | 0.412 | 0.42 | 0.416 | 0.765 |
| 10 mg/ml, | | | | |
| 10K, (F)** | | | | |
| Piperacillin, 1 mg/ml, | 0.245 | 0.241 | 0.243 | 0.447 |
| 10K, | | | | |
| (R)* | | | | |
| Piperacillin, | 0.072 | 0.069 | 0.071 | 0.130 |
| 1 mg/ml, 10K, | | | | |
| (F)** | | | | |

*(R) is the retentate (retained on the filter)
**(F) in the filtrate

### EXAMPLE 4

### 3K(3 kD ) Filter

| | | | | Index of |
|---|---|---|---|---|
| Experimental | | | | Experimental |
| Samples | OD1 | OD2 | Mean OD | Samples |
| Piperacillin, 10 mg/ml, | 2.311 | 2.444 | 2.378 | 4.370 |
| 3K, (R)* | | | | |
| Piperacillin, 10 mg/ml, | 0.031 | 0.033 | 0.032 | 0.059 |
| 3K, (F)** | | | | |
| Piperacillin, 1 mg/ml, | 0.476 | 0.5 | 0.488 | 0.897 |
| 3K, (R)* | | | | |
| Piperacillin, | 0.041 | 0.04 | 0.041 | 0.074 |
| 1 mg/ml, 3K, | | | | |
| (F)** | | | | |

*(R) is the retentate (retained on the filter)
**(F) in the filtrate

### EXAMPLE 5

The experimental activity consisted on: (1) formulating a ZOSYN® bulk product using a batch size of 10 L, (2) filtering the bulk solution through a filter with a porosity size of, at least 5 μm, and (3) removing the galactomannan content from the bulk solution by ultra-filtration/diafiltration technique. Sampling process was conducted during the ultra-filtration treatment of the bulk solution for up to ten concentration (10×) and six diafiltration (6 DV) processes.

Bulk Formulation

A bulk solution of a development batch of Zosyn® bulk product was formulated at a concentration of 250 mg/mL Piperacillin and 31.25 mg/mL Tazobactam with a 2% excess of Piperacillin to drive the reaction to completion. Piperacillin Monohydrate (PMH) raw material, lot number 2000084742, which tested positive to galactomannan (GM) (using the Bio-Rad Platelia™ EIA kit) was used in this study. Sodium Bicarbonate (limiting reagent) was added on a stoichiometric basis. The total batch size was of 10 L. The weighting data is summarized in Raw Materials Table. Bulk formulation was performed well, as expected. For protocol purpose, the product bulk solution was not brought to the final volume (Qs). The reaction was considered completed since the solution reached a pH of 6.0 (acceptable pH limit of 6.8 or less). A bulk product volume of about eight liters (8 L) was obtained prior to qs. For the purpose of this study, the product bulk solution was not brought to the final volume. The Raw Materials Table summarizes the different formulation ingredients used for the manufacture of the experimental batch.

Raw Materials Table

| | | | Expected | Actual |
|---|---|---|---|---|
| Material | Lot Number | Supplier | Weight^{a}, kg | Weight^{b}, kg |
| Piperacillin | 2000084742 | BMS^{c} | 2.6956 | 2.6956 |
| Monohydrate, | | | | |
| USP | | | | |
| Tazzobactam | 3K78 | Otsuka | 0.3125 | 0.3125 |
| Sodium | C3-01527 | Fisher | 0.4932 | 0.4932 |
| Bicarbonate, USP | | Scientific | | |

^{a}Expected weights were calculated using the corresponding equations included in Protocol CR-0169/04.
^{b}Materials were weighed in bench scale, number C1833A.
^{c}BMS is Bristol-Myers Squibb

Filtration

Once the reaction was completed and prior to reach the final volume of 10 L, bulk product was filtered through a nylon membrane filter of 0.2 μm porosity size (CUNO® LifeASSURE™ capsule).

Ultrafiltration/Diafiltration Process

The ultrafiltration (UF) filtering process was conducted by using a 5-kD Omega® membrane (Part #OS005G02). Above membrane size was selected since the GM removal efficiency is greater than the 1, 3, and 10 kD membranes.

A total volume of 6L ZOSYN® bulk solution was used to evaluate the operational efficiency of the filtration system. The UF system operated with a feed pressure of 37 psi (42 psi, maximum pressure) and a retentate pressure of 35 psi (39 psi, maximum pressure). During the ultrafiltration, permeate pool samples were taken at 2×, 4×, 8×, and 10× concentration. Once the 10× concentration was achieved, a recovery yield of 96% for Piperacillin and 86% for Tazobactam was obtained as shown in Table A.

A diafiltration filtering process followed and was executed by completing six diafiltration volumes (2 DV, 4 DV, 5 DV, and 6 DV). Collected data demonstrated that, after four diafiltration volumes (4 DV), a 100% recovery yield is obtained for both Piperacillin and Tazobactam as shown in Table B.

TABLE A
Mass Balance for Ultrafiltration

| | | | Mass | Progressive | | Mass | Progressive |
|---|---|---|---|---|---|---|---|
| | | | Balance | Yield | | Balance | Yield |
| | Volume, | Piperacillin, | Piperacillin, | (Piperacillin), | Tazobactam, | Tazobactam, | (Tazobactam), |
| Sample | L | mg/mL | g | % | mg/mL | g | % |
| Feed | 6.0 | 305.6 | 1833.6 | N/A | 37.358 | 224.1 | N/A |
| Pool- | | | | | | | |
| Initial | | | | | | | |
| Control | | | | | | | |
| Permeate | 3.0 | 277.9 | 833.7 | 45 | 35.337 | 106.0 | 47 |
| Pool 2X | | | | | | | |
| Permeate | 4.5 | 286.5 | 1289.3 | 70 | 35.789 | 161.1 | 72 |
| Pool 4X | | | | | | | |
| Permeate | 5.25 | 290 | 1522.5 | 83 | 36.115 | 189.6 | 85 |
| Pool 8X | | | | | | | |
| Permeate | 5.4 | 324.6 | 1752.8 | 96 | 35.543 | 191.9 | 86 |
| Pool | | | | | | | |
| 10X | | | | | | | |
| Feed | 0.6 | 287.6 | 172.6 | N/A | 35.833 | 21.5 | N/A |
| Pool | | | | | | | |
| 10X | | | | | | | |

TABLE B
Mass Balance for Diafiltration

| | | | Mass | Progressive | | Mass | Progressive |
|---|---|---|---|---|---|---|---|
| | | | Balance | Yield | | Balance | Yield |
| | Volume, | Piperacillin, | Piperacillin, | (Piperacillin), | Tazobactam, | Tazobactam, | (Tazobactam), |
| Sample | L | mg/mL | g | % | mg/mL | g | % |
| Permeate | 6.6 | 272.0 | 1795.2 | 98 | 33.596 | 221.7 | 99 |
| Pool | | | | | | | |
| 2DV | | | | | | | |
| Permeate | 7.8 | 236.8 | 1847.0 | 101 | 29.131 | 227.2 | 101 |
| Pool | | | | | | | |
| 4DV | | | | | | | |
| Permeate | 8.4 | 218.4 | 1834.6 | 100 | 26.979 | 226.6 | 101 |
| Pool | | | | | | | |
| 5DV | | | | | | | |
| Feed | 0.6 | 20.1 | 12.1 | N/A | 1.740 | 1.0 | N/A |
| Pool | | | | | | | |
| 6DV | | | | | | | |
| Permeate | 9.0 | 202.0 | 1818.0 | 99 | 24.949 | 224.5 | 100 |
| Pool | | | | | | | |
| 6DV | | | | | | | |
| Permeate | 10.0 | 184.0 | 1840.0 | 100 | 22.661 | 226.6 | 101 |
| Pool - qs | | | | | | | |
| solution | | | | | | | |

Concurrently, GM detection testing was performed on each sample included in Tables A and B. The results obtained for the GM detection test are displayed in Table C.

TABLE C
Galactomannan Bio-Rad Platelia ™ Testing Results

| | | | | | | Galacto- |
|---|---|---|---|---|---|---|
| | | | | | | mannan |
| | | Optical | Optical | Average | | Results |
| | Dilution | Density - | Density - | Optical | | (Positive/ |
| Sample | Factor | test #1 | test #2 | Density | Index | Negative) |
| Feed | 10X | 3.149 | 3.149 | 3.149 | 4.386 | Positive |
| Pool - | | | | | | |
| Initial | | | | | | |
| Control | | | | | | |
| Feed | 100X | 1.062 | 0.925 | 0.994 | 1.384 | Positive |
| Pool - | | | | | | |
| Initial | | | | | | |
| Control | | | | | | |
| Permeate | 10X | 0.085 | 0.084 | 0.085 | 0.118 | Negative |
| Pool 2X | | | | | | |
| Permeate | 100X | 0.044 | 0.052 | 0.048 | 0.067 | Negative |
| Pool 2X | | | | | | |
| Permeate | 10X | 0.123 | 0.138 | 0.131 | 0.182 | Negative |
| Pool 4X | | | | | | |
| Permeate | 100X | 0.052 | 0.121 | 0.087 | 0.120 | Negative |
| Pool 4X | | | | | | |
| Permeate | 10X | 0.116 | 0.102 | 0.109 | 0.152 | Negative |
| Pool 8X | | | | | | |
| Permeate | 100X | 0.074 | 0.047 | 0.061 | 0.084 | Negative |
| Pool 8X | | | | | | |
| Permeate | 10X | 0.086 | 0.086 | 0.086 | 0.120 | Negative |
| Pool 10X | | | | | | |
| Permeate | 100X | 0.045 | 0.044 | 0.045 | 0.062 | Negative |
| Pool 10X | | | | | | |
| Feed | 10X | Over | Over | Over | Over | Positive |
| Pool 10X | | | | | | |
| Feed | 100X | 3.319 | 3.377 | 3.348 | 4.663 | Positive |
| Pool 10X | | | | | | |
| Permeate | 10X | 0.134 | 0.094 | 0.114 | 0.159 | Negative |
| Pool | | | | | | |
| 2DV | | | | | | |
| Permeate | 100X | 0.052 | 0.055 | 0.054 | 0.075 | Negative |
| Pool | | | | | | |
| 2DV | | | | | | |
| Permeate | 10X | 0.106 | 0.146 | 0.126 | 0.175 | Negative |
| Pool | | | | | | |
| 4DV | | | | | | |
| Permeate | 100X | 0.050 | 0.064 | 0.057 | 0.079 | Negative |
| Pool | | | | | | |
| 4DV | | | | | | |
| Permeate | 10X | 0.104 | 0.121 | 0.113 | 0.157 | Negative |
| Pool | | | | | | |
| 5DV | | | | | | |
| Permeate | 100X | 0.057 | 0.049 | 0.053 | 0.074 | Negative |
| Pool | | | | | | |
| 5DV | | | | | | |
| Feed | 10X | Over | Over | Over | Over | Positive |
| Pool | | | | | | |
| 6DV | | | | | | |
| Feed | 100X | 2.830 | 2.712 | 2.771 | 3.859 | Positive |
| Pool | | | | | | |
| 6DV | | | | | | |
| Permeate | 10X | 0.120 | 0.103 | 0.112 | 0.155 | Negative |
| Pool | | | | | | |
| 6DV | | | | | | |
| Permeate | 100X | 0.045 | 0.047 | 0.046 | 0.064 | Negative |
| Pool | | | | | | |
| 6DV | | | | | | |
| Permeate | 10X | 0.103 | 0.119 | 0.111 | 0.155 | Negative |
| Pool - qs | | | | | | |
| solution | | | | | | |
| Permeate | 100X | 0.059 | 0.049 | 0.054 | 0.075 | Negative |
| Pool - qs | | | | | | |
| solution | | | | | | |

Note: 0.718, Cut-off Control Average OD

Permeate pool samples gave negative results for galactomannan. All testing results were well within the established specifications.

1. A pharmaceutical composition comprising effective amounts of (a) piperacillin or a pharmaceutically acceptable salt thereof, (b) tazobactam or a pharmaceutically acceptable salt thereof substantially free of galactomannan or a pharmaceutically acceptable salt thereof.

2. A pharmaceutical composition according to claim 1 wherein the piperacillin is piperacillin sodium.

3. A pharmaceutical composition according to claim 1 wherein the tazobactam is tazobactam sodium.

4. A pharmaceutical composition according to any one of claim 1 wherein the composition is a lyophilized powder.

5. A method for the treatment or control of bacterial infections in a mammal wherein the method comprises administering to said mammal a therapeutically effective amount of the pharmaceutical composition of claim 1.

6. A process for preparing a lyophilized pharmaceutical composition substantially free of galactomannan which comprises the steps of:
a) dissolving piperacillin, and tazobactam, in an aqueous solvent forming a solution and adjusting the pH to about 6.5;
b) filtering the solution through a cut off filter;
c) collecting a filtrate;
d) cooling the filtrate to a temperature below −35° C. in a lyophilizer;
e) evacuating the lyophilizer to a pressure of about 300 μM Hg (micrometers of mercury) (40 pascals) and heating the lyophilizer to about +5° C.;
f) maintaining the temperature and pressure for a sufficient time to remove water from the aqueous solvent forming a lyophilized solid;
g) drying the lyophilized solid at about +45° C.

7. A pharmaceutical composition according to claim 6 wherein the cut off filter is about 3 kD molecular weight to about 10 kD molecular weight.

8. A pharmaceutical composition according to claim 6 wherein the cut-off filter is about 3 kD mw.

9. A pharmaceutical composition according to claim 6 wherein the cut-off filter is about 5 kD mw.

10. A pharmaceutical composition according to claim 6 further comprising an index of experimental samples of the collected filtrate to be less than 0.5.

11. A process for the manufacture of a pharmaceutical composition in the form of a powder that can be reconstituted by addition of a compatible reconstitution diluent prior to administration to a mammal or in the form of a frozen composition which when thawed can be diluted with a compatible diluent prior to administration to a mammal which process comprises freezing or freeze-drying a solution substantially free of galactomannan containing effective amounts of (a) piperacillin or a pharmaceutically acceptable salt thereof, (b) tazobactam or a pharmaceutically acceptable salt thereof or a pharmaceutically acceptable salt thereof in an aqueous vehicle.

12. A pharmaceutical composition comprising an effective amount of piperacillin substantially free of galactomannan or a pharmaceutically acceptable salt thereof.

13. A pharmaceutical composition according to claim 12 wherein the piperacillin is piperacillin sodium.
